(19) United States
(12) Patent Application Publication
(10) Pub. No.: US 2007/0255396 A1
(43) Pub. Date: Nov. 1, 2007

(54) Chrodae Tendinae Girdle

(75) Inventors: Nareak Douk, Lowell, MA (US); Nasser Rafiee, Andover, MA (US); Vincent J. Cangialosi, Beverly, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/560,983

(22) PCT Filed: Jun. 18, 2004
(86) PCT No.: PCT/US04/19717

§ 371 Date: Mar. 19, 2007

Related U.S. Application Data
(60) Provisional application No. 60/480,364, filed on Jun. 20, 2003.

(51) Int. Cl. A61F 2/24

(52) U.S. Cl. 623/2.1

(57) ABSTRACT

A girdle for surrounding the chordae tendinae of a heart valve, and a system and method for delivering the girdle. The girdle gathers the chordae tendinae into a bundle to effectively shorten the chordae tendinae to resolve or reduce valve leaflet prolapse. The system includes a girdle releaseably carried within a delivery catheter, and a push rod to release the girdle from the delivery catheter. The girdle has a filamentous linear delivery configuration and one of several annular treatment configurations. The girdle may have a locking mechanism for locking the girdle in an annular treatment configuration.

### RELATED APPLICATION

This application claims priority to U.S. Provisional Application No. 60/480,364, “Method and System for Reducing Mitral Valve Regurgitation” to Nareak Douk and Nasser Rafiee, filed Jun. 20, 2003, the entirety of which is incorporated by reference.

### TECHNICAL FIELD

The technical field of this disclosure is medical devices, particularly, heart valve repair systems and method of using the same.

### BACKGROUND OF THE INVENTION

Heart valves, such as the mitral valve, are sometimes damaged by disease or by aging, which can cause problems with the proper function of the valve. Heart valve problems generally take one of two forms: stenosis, in which a valve does not open completely or the opening is too small, resulting in restricted blood flow; or insufficiency or regurgitation, in which blood leaks backward across a valve that should be closed. Valvular insufficiency may result from a dilated valve annulus, because of heart disease. Alternatively, regurgitation may be caused by mitral valve prolapse, which is considered a genetic disorder rather than a conventional disease. Valve replacement may be required in severe cases to restore cardiac function.

Any one or more of the mitral valve structures, i.e., the anterior and posterior leaflets, the chordae, the papillary muscles or the annulus may be compromised genetically, or by damage from disease or injury, causing the mitral valve insufficiency. Mitral valve regurgitation may occur as the result of the leaflets being moved back from each other by the dilated annulus, or by the valve leaflets prolapsing beyond the valve annulus into the atrium. Thus, without correction, the mitral valve insufficiency may lead to disease progression and/or further enlargement and worsening of the insufficiency. In some instances, correction of the regurgitation may not require repair of the valve leaflets themselves, but simply a reduction in the size of the annulus.

A variety of techniques have been attempted to reduce the diameter of the mitral annulus and eliminate or reduce valvular regurgitation in patients with incompetent valves. Current surgery to correct mitral regurgitation in humans includes a number of mitral valve replacement and repair techniques.

Valve replacement can be performed through open-heart surgery, open chest surgery, or percutaneously. The native valve is removed and replaced with a prosthetic valve, or a prosthetic valve is placed over the native valve. The valve replacement may be a mechanical or a biological valve prosthesis. The open chest and percutaneous procedures avoid opening the heart and cardiopulmonary bypass. However, the valve replacement may result in a number of complications including a risk of, endocarditis. Additionally, mechanical valve replacement requires subsequent anticoagulation treatment to prevent thromboembolisms.

As an alternative to valve replacement, various surgical valve repair techniques have been used including quadrangular segmental resection of a diseased posterior leaflet; transposition of posterior leaflet chordae to the anterior leaflet; valvuloplasty with plication and direct suturing of the native valve; substitution, reattachment or shortening of chordae tendinae; and annuloplasty in which the effective size of the valve annulus is contracted by attaching a prosthetic annuloplasty ring to the endocardial surface of the heart around the valve annulus. The annuloplasty techniques may be used in conjunction with other repair techniques. Typically, such rings are sutured along the posterior mitral leaflet adjacent to the mitral annulus in the left atrium. The rings either partially or completely encircle the valve, and may be rigid or flexible/non-elastic. All of these surgical procedures require cardiopulmonary bypass, though some less and minimally invasive techniques for valve repair and replacement are being developed.

Although mitral valve repair and replacement can successfully treat many patients with mitral valve insufficiency, techniques currently in use are attended by significant morbity and mortality. Most valve repair and replacement procedures require a thoractomy, to gain access into the patient's thoracic cavity. Surgical intervention within the heart generally requires isolation of the heart and coronary blood vessels from the remainder of the arterial system and arrest of cardiac function. Open chest techniques with large sternum openings are typically used. Those patients undergoing such techniques often have scarring retraction, tears or fusion of valve leaflets as well as disorders of the subvalvular apparatus.

Recently other surgical procedures have been provided to reduce the mitral annulus using a less invasive surgical technique. According to this method, a prosthesis is transvenously advanced into the coronary sinus and the prosthesis is deployed within the coronary sinus to reduce the diameter of the mitral annulus. This may be accomplished in an open procedure or by percutaneously accessing the venous system by one of the internal jugular, brachial, radial, or femoral veins. The prosthesis is tightened down within the coronary sinus, located adjacent the mitral annulus, to reduce the mitral annulus.

While the coronary sinus implant provides a less invasive treatment alternative, the placement of the prosthesis within the coronary sinus may be problematic for a number of reasons. Sometimes the coronary sinus is not accessible. The coronary sinus on a particular individual may not wrap around the heart far enough to allow enough encircling of the mitral valve. Also, leaving a device in the coronary sinus may result in formation and breaking off of thrombus that may pass into the right atrium, right ventricle and ultimately the lungs causing a pulmonary embolism. Another disadvantage is that the coronary sinus is typically used for placement of a pacing lead, which may be precluded with the placement of the prosthesis in the coronary sinus.

It would be desirable, therefore, to provide a method and device for reducing mitral valve regurgitation that would overcome these and other disadvantages.

### SUMMARY OF THE INVENTION

One aspect of the present invention provides a girdle for surrounding the chordae tendinae of a diseased heart valve. The girdle effectively shortens the chordae tendinae to resolve or reduce valve leaflet prolapse. The girdle has a filamentous linear delivery configuration. The girdle may have one of several annular treatment configurations. The girdle is elastically deformable between an annular treatment configuration and the linear delivery configuration. In one embodiment, the girdle has a shape memory of the annular treatment configuration. In another embodiment, the girdle is locked into position surrounding the chordae tendinae with a locking mechanism.

A system of the present invention includes a girdle for surrounding the chordae tendinae of a diseased heart valve. The girdle is releaseably carried within a delivery catheter, which has a push rod to release the girdle from the catheter.

Another aspect of the present invention provides a method for treating a diseased heart valve. The method comprises delivering a self-forming annular girdle in a lumen of a catheter proximate the diseased heart valve, releasing the self forming annular girdle and encircling chordae tendinae of the diseased heart valve with the girdle.

The foregoing and other features and advantages of the invention will become further apparent from the following detailed description of the presently preferred embodiments, read in conjunction with the accompanying drawings, which are not to scale. The detailed description and drawings are merely illustrative of the invention, rather than limiting the scope of the invention being defined by the appended claims and equivalents thereof.

### BRIEF DESCRIPTION OF THE DRAWINGS

FIG. 1 shows a detailed illustration of one embodiment of a heart valve repair system including a chordae tendinae girdle in accordance with the present invention.

FIG. 2 shows one embodiment of a girdle of the heart valve repair system illustrated in FIG. 1 in accordance with the present invention.

FIG. 3 shows another embodiment of a girdle of the heart valve repair system illustrated in FIG. 1 in accordance with the present invention.

FIG. 4 shows another embodiment of a girdle of the heart valve repair system illustrated in FIG. 1 in accordance with the present invention.

FIG. 5 shows another embodiment of a girdle of the heart valve repair system illustrated in FIG. 1 in accordance with the present invention.

FIG. 6 shows another embodiment of a girdle of the heart valve repair system illustrated in FIG. 1 in accordance with the present invention.

FIG. 7 shows another embodiment of a girdle of the heart valve repair system illustrated in FIG. 1 in accordance with the present invention.

FIG. 8 shows another embodiment of a girdle of the heart valve repair system illustrated in FIG. 1 in accordance with the present invention.

FIG. 9 shows one embodiment of a heart valve repair system inserted percutaneously in accordance with the present invention.

FIGS. 10 to 14 show the progression of the placement of one embodiment of the girdle around the chordae tendinae in accordance with the present invention.

FIG. 15 shows the girdle of FIG. 3 placed about the chordae tendinae.

FIG. 16 shows the girdle of FIG. 4 placed about the chordae tendinae.

FIG. 17 shows the girdle of FIG. 5 placed about the chordae tendinae.

FIG. 18 shows the girdle of FIG. 7 placed about the chordae tendinae.

FIG. 19 shows a detailed illustration of another embodiment of a heart valve repair system including a chordae tendinae girdle in accordance with the present invention.

FIG. 20 shows one embodiment of a girdle of the heart valve repair system illustrated in FIG. 19 in accordance with the present invention.

FIG. 21 shows a detailed illustration of another embodiment of a heart valve repair system including a chordae tendinae girdle in accordance with the present invention.

FIG. 22 shows one embodiment of a girdle of the heart valve repair system illustrated in FIG. 21 in accordance with the present invention.

FIG. 23 shows a flow chart for a method of using a heart valve repair system in accordance with the present invention.

### DETAILED DESCRIPTION OF THE PRESENTLY PREFERRED EMBODIMENT

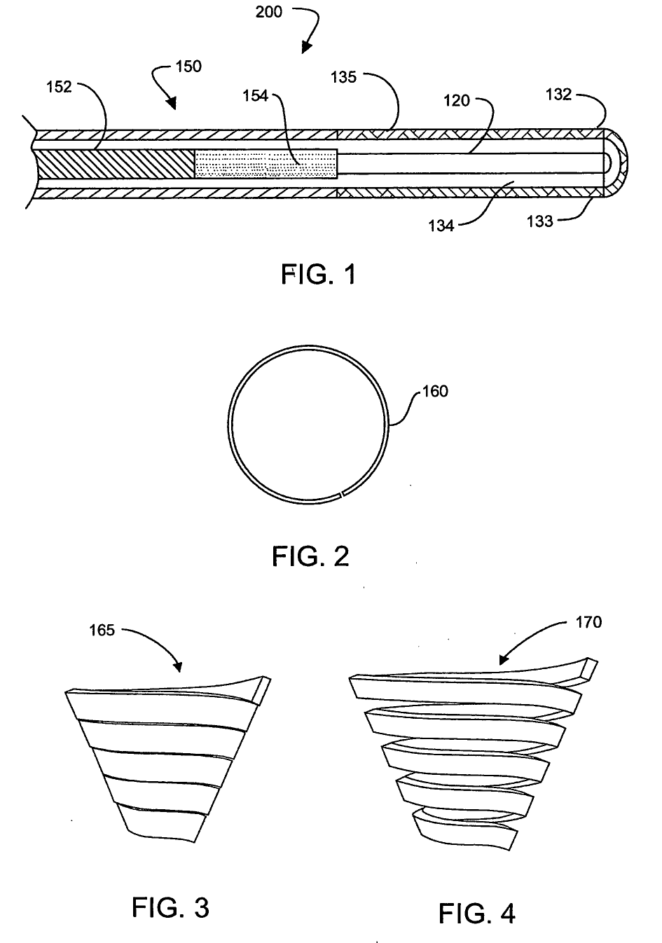

FIG. 1 shows a detailed illustration of a heart valve repair system 200. Heart valve repair system 200 comprises an elongate delivery device having a delivery catheter 132 and push rod 150. Delivery catheter 132 includes lumen 134 and distal end 133. System 200 further includes girdle 120 disposed within lumen 134 of delivery catheter 132. In one embodiment, push rod 150 includes rigid proximal portion 152 and flexible distal portion 154. Flexible portion 154 contacts girdle 120. In one embodiment, push rod 150 is moved in an axial direction to push girdle 120 from delivery catheter 132. Elongate push rod 150 may be solid or a hollow rod closed at its distal end for contact with girdle device 120. Push rod 150 may be composed of any material that is sufficiently flexible to traverse a tortuous path to the left ventricle, and sufficiently incompressible to controllably push girdle 120 out of delivery catheter 132. Examples of suitable plastic materials to fabricate push rod 150 include amides, polyimides, polyolefins, polyesters, urethanes, thermoplastics, thermoset plastics, and blends, laminates or copolymers thereof. Push rod 150 may also be composed of metal, such as a core wire with a coiled spring at the distal end. Push rod 150 may also have a lubricious coating on the outer surface to provide lubrication between the inner surface of delivery catheter 132 and the outer surface of push rod 150.

Delivery catheter 132 may include reinforced portion 135 to help maintain girdle 120 in its deformed linear delivery configuration. Reinforced portion 135 may incorporate a braided material or other stiffening member. In another embodiment, reinforced portion 135 may comprise a pre-shaped curve to assist in accurately placing girdle 120 within the patient's cardiac anatomy. A thermoplastic material can be used in reinforced portion 135 to form and retain the pre-shaped curve.

Girdle 120 is held within delivery catheter 132 in a linear delivery configuration so that it may be delivered via catheter 132 to the chordae tendinae. The linear delivery configuration is obtained by deforming girdle 120 from its annular treatment configuration and inserting the linear deformed girdle into the delivery catheter 132. Girdle 120 can be deformed into the delivery configuration before or during insertion into the delivery catheter 132. Girdle 120 may be composed of a biocompatible material having sufficient elastic properties to permit deformation from the annular treatment configuration into the linear delivery configuration and subsequent re-formation of the device back into the annular treatment configuration. In one embodiment, girdle 120 may be composed of a biocompatible metal such as nitinol, stainless steel, or cobalt-based alloys such as MP35N® from SPS Technology Inc. or Elgiloy® from Elgiloy Specialty Metals. Biocompatible engineering plastics may also be used, such as amides, polyimides, polyolefins, polyesters, urethanes, thermoplastics, thermoset plastics, and blends, laminates or copolymers thereof.

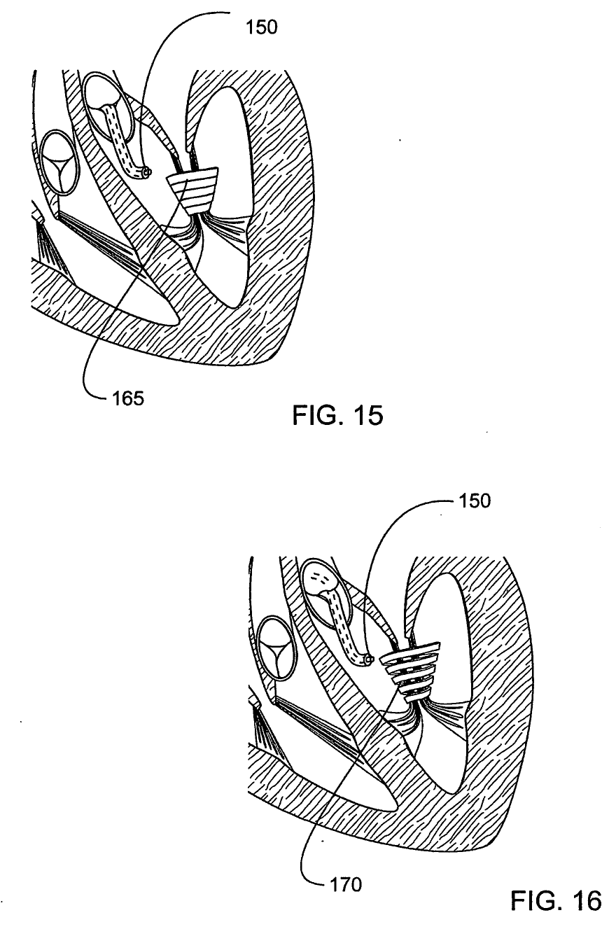

FIGS. 2 to 8 illustrate several embodiments of girdle 120. FIG. 2 illustrates girdle 160 having a filamentous body that forms a circular ring when fully deployed. The filamentous body may have a round or other cross-section. FIGS. 3 and 15 illustrate girdle 165 having a hollow frusto-conical shape when deployed. Girdle 165 is composed of a flat or round wire, or other filamentous material, formed into a closed coil and heat set. The closed coil may be formed by wrapping the wire around a mandrel or other device suitable for forming the cone-shape. Heat setting the formed coil provides shape memory to the material so that girdle 165 will return to the annular treatment configuration from the deformed linear delivery configuration when girdle 165 is delivered to more than one chorda tendina, possibly all of the chordae tendinae. In another embodiment, the closed coil of girdle 165 may be formed by first creating a cone from a sheet of material and then cutting the cone in a spiraling manner to form filaments of the coil. The coil may be cut using a laser or any other suitable cutting method. The cut coil may be heat set, if necessary, to provide the desired shape memory to girdle 165.

FIGS. 4 and 16 illustrate girdle 170 that also forms a hollow frusto-conical shape when deployed. Girdle 170 may be formed from materials similar to those discussed above for girdle 120. Girdle 170 may be formed in a manner similar to that of girdle 165, however, the coil of girdle 170 forms an open coil around the chordae tendinae as illustrated in FIG. 16.

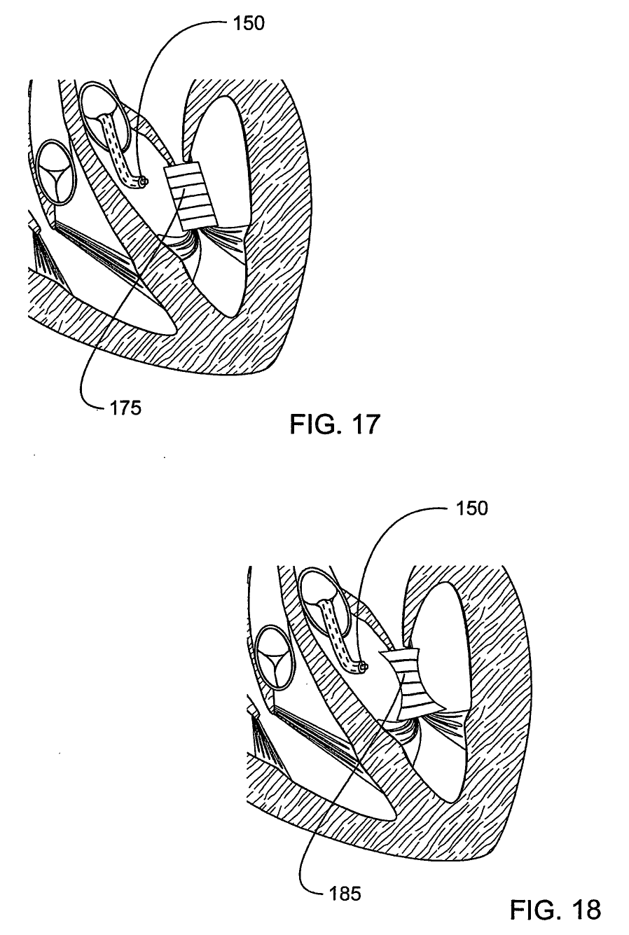

FIGS. 5 and 17 illustrate another embodiment of a girdle 175 of the heart valve repair system illustrated in FIG. 1. Girdle 175 forms a hollow cylinder when deployed. Girdle 175 may be formed from material similar to those discussed above for girdle 120. Girdle 175 may be formed in a manner similar to that of girdle 165, however, the coil of girdle 175 forms a closed coil around the chordae tendinae as illustrated in FIG. 17.

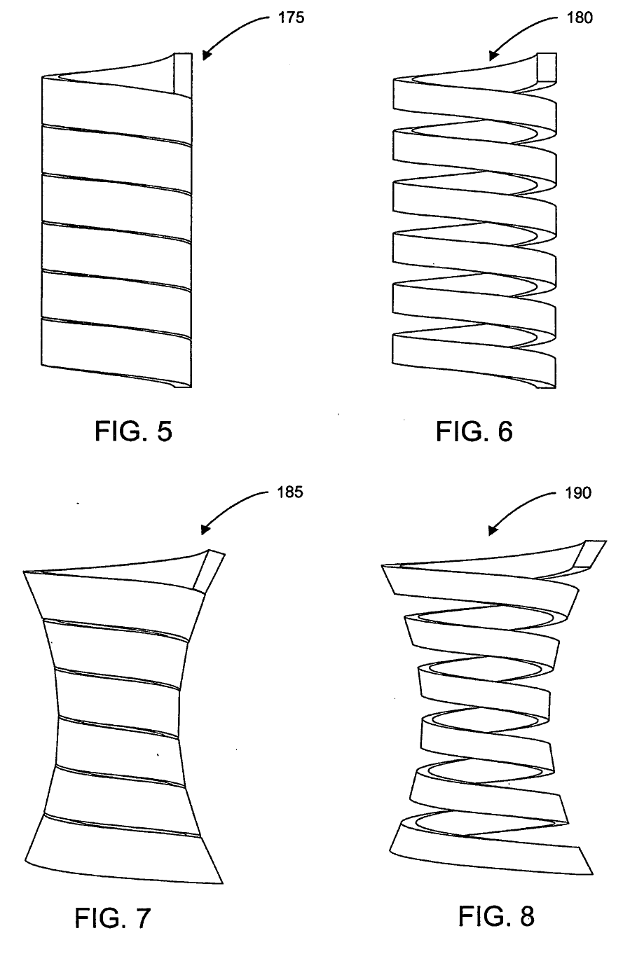

FIG. 6 illustrates girdle 180 that also forms a hollow cylinder when deployed. Girdle 180 may be formed from material similar to those discussed above for girdle 120. Girdle 180 may be formed in a manner similar to that of girdle 175; however, the coil of girdle 180 forms an open coil around the chordae tendinae.

FIGS. 7 and 18 illustrate another embodiment of a girdle 185 of the heart valve repair system illustrated in FIG. 1. Girdle 185 forms a hollow hourglass shape when deployed. Girdle 185 may be formed from material similar to those discussed above for girdle 120. Girdle 185 may be formed in a manner similar to that of girdle 165, however, the coil of girdle 185 forms an hourglass-shaped closed coil around the chordae tendinae as illustrated in FIG. 18.

FIG. 8 illustrates girdle 190 that forms a hollow hourglass shape when deployed. Girdle 190 may be formed from material similar to those discussed above for girdle 120. Girdle 190 may be formed in a manner similar to that of girdle 185; however, the coil of girdle 190 forms an hourglass-shaped open coil around the chordae tendinae.

Those with skill in the art will recognize that the lengths and transverse dimensions of girdles 165, 170, 175, 180, 185 and 190 may be selected to accommodate the size and shape of a specific patient's heart structure.

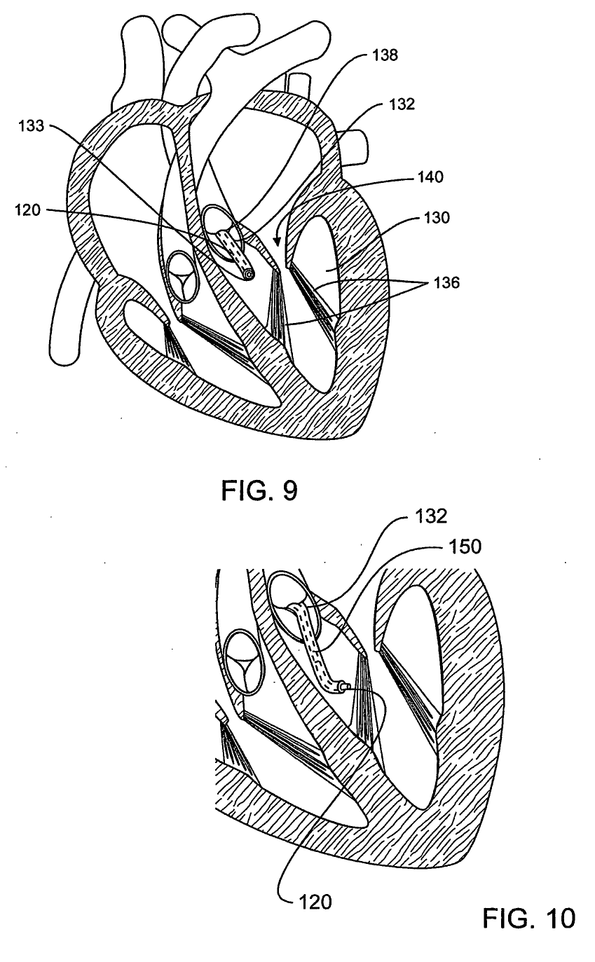

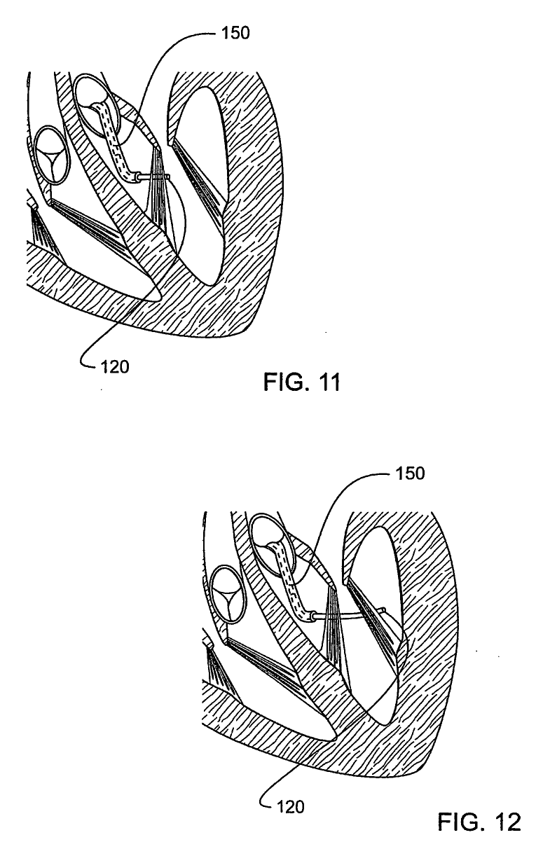

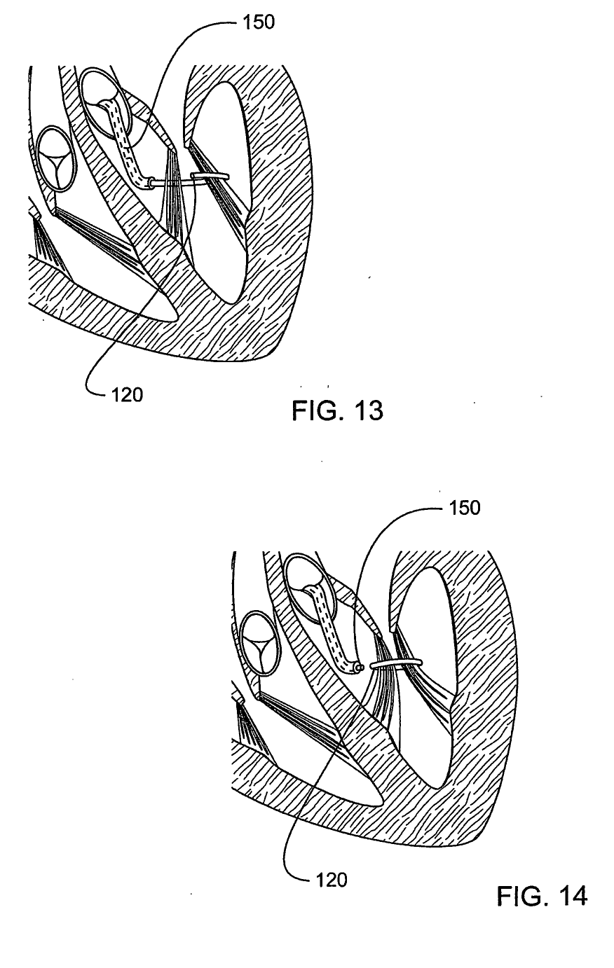

FIGS. 9 to 14 illustrate the deployment of girdle 120 into an annular treatment configuration around chordae tendinae 136 of the mitral valve. As illustrated in FIG. 9, delivery catheter 132 has been advanced transluminally through the patient's vasculature and through aortic valve 138 into the left ventricle. Those with skill in the art will recognize that the devices and methods disclosed herein may be applied alternatively to the chordae tendinae within the right ventricle. FIG. 9 shows one embodiment of a heart valve repair system wherein girdle 120 is held in a deformed linear delivery configuration within an elongate delivery element. The collapsible girdle can be delivered via a percutaneous transluminal route, using a catheter. Alternatively, the girdle can be delivered surgically, using a cannula, a trocar or an endoscope as the elongate delivery element.

For the exemplary case of the heart valve repair system shown in FIGS. 9-14, an elongate element having lumen 134 is first placed to provide a path from the exterior of the patient to left ventricle 130. In one embodiment, the elongate element is catheter 132. Girdle 120 can then be advanced through lumen 134 so that girdle 120 is located at the mitral valve chordae tendinae 136 for deployment. FIG. 9 illustrates an aortic approach to the left ventricle: catheter 132 may be inserted into a femoral artery, through the aorta, through aortic valve 138 and into left ventricle 130. Those skilled in the art will appreciate that alternative paths are available to gain access to the left ventricle. For surgical approaches with an open chest, the elongate delivery element can be a trocar or cannula inserted directly in the aortic arch. The elongate delivery element can then follow the same path as in the percutaneous procedure to reach the left ventricle. The left ventricle can also be accessed transluminally through the patient's venous system to the right ventricle, then using known trans-septal techniques to traverse the ventricular septum. Related transluminal or surgical approaches can be used to access the chordae tendinae of the tricuspid valve.

As shown in FIG. 9, delivery catheter 132 is advanced until distal end 133 is adjacent chordae tendinae 136 of the mitral valve. The advancement of delivery catheter 132 to the chordae tendinae may be monitored by methods known in the art such as fluoroscopy and ultrasonography. In one embodiment, delivery catheter 132 and/or push rod 150 may include radiopaque markers to improve fluoroscopic visualization of the component. To deploy girdle 120, push rod 150 is advanced towards distal end 133 of delivery catheter 132.

As illustrated in the series of FIGS. 9 to 14, the continued advancement of push rod 150 extends more of girdle 120 out of catheter 132, and, due to the elastic shape memory of the girdle material, girdle 120 begins to form ring 160 around the chordae tendinae. Upon complete deployment, girdle 120 surrounds the chordae tendinae to form ring 160. In another technique, girdle 120 is deployed to form the annular treatment configuration by holding push rod 150 in position while retracting delivery catheter 132. In this technique, girdle 120 will reform into the annular treatment configuration as delivery catheter 132 is withdrawn in a proximal direction.

Once formed, the inner diameter of ring 160 contacts the chordae tendinae. Further, the inner diameter of the ring 160 is sized to draw the chordae tendinae closer together to form a bundle to effectively achieve chordal shortening. This shortening of the chordae tendinae resolves or reduces valve leaflet prolapse. Further, the placement of the girdle simulates surgical techniques such as chordal transposition or papillary muscle repositioning. In some applications, the tension that the girdle provides in the chordae tendinae may reduce the diameter of the mitral valve annulus, resulting in more complete closing of the leaflets to eliminate valve regurgitation.

FIGS. 15 to 18 illustrate girdles 165, 170, 175, 185 (shown in FIGS. 3, 4, 5 and 7, respectively) deployed in the annular treatment configuration. As illustrated, each girdle surrounds and gathers the chordae tendinae to form a bundle to effectively achieve a degree of chordal shortening.

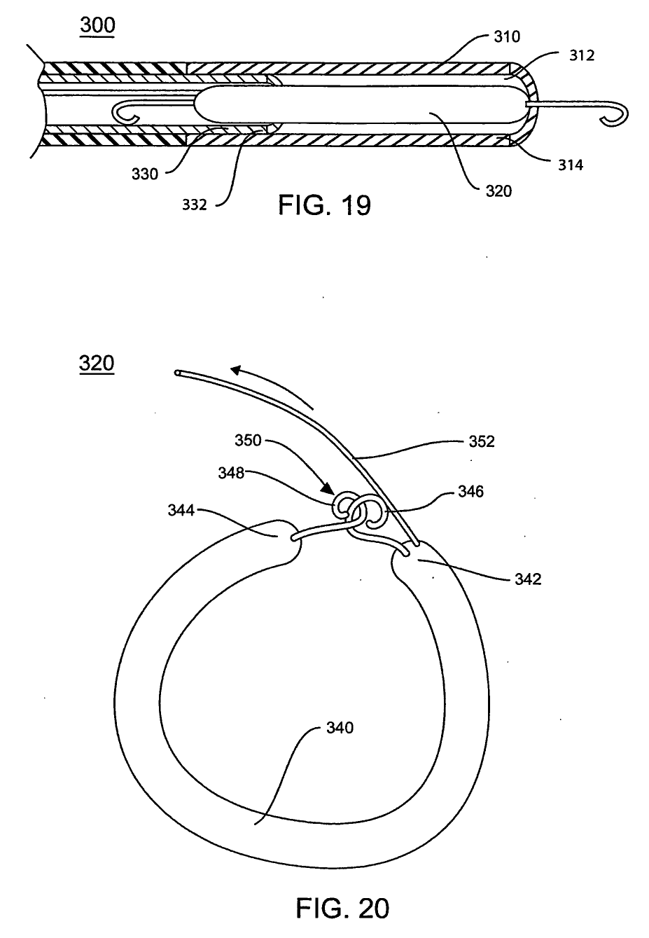

FIGS. 19 and 20 illustrate another embodiment of heart valve repair system 300 made in accordance with the present invention. Heart valve repair system 300 comprises delivery catheter 310, girdle 320 and secondary catheter 330. Delivery catheter 310 includes lumen 312 and distal end 314. Secondary catheter 330 is disposed within lumen 312 of delivery catheter 310. Girdle 320 is disposed within secondary catheter 330. Secondary catheter 330 may be composed of a thermoplastic or other shape memory material. In one embodiment, secondary catheter 330 includes shape memory such that the secondary catheter curves around the chordae tendinae when extended from delivery catheter 310.

Girdle 320 comprises elongate body 340 for forming a girdle and locking mechanism 350 to hold the girdle in the desired position around the chordae tendinae. Elongate body 340 has first end 342 and second end 344 that are drawn together to form the girdle. Elongate body 340 may be composed of biocompatible elastic or inelastic material, and may be a flat strap or a filament that is round in cross-section. Elongate body 340 may be composed of elastic materials such as natural rubber, synthetic rubber, polyurethane, thermoplastic elastomer or the like. Such elastic materials may allow girdle 320, and other embodiments of the invention, to expand and contract with the natural movement of the chordae tendinae while still effectively shortening the length of the chordae tendinae. Locking mechanism 350 is comprised of first hook 346 located at first end 342 and second hook 348 located at second end 344. Hooks 346 and 348 may be attached to elongate body 340 by insert molding, adhesive or mechanical bond. Heart valve repair system 300 further includes tether 352 releaseably attached adjacent end 342 of elongate body 340. Tether 352 may be releaseably attached to elongate body 340 via a sacrificial joint. In one embodiment, tether 352 includes a weakening near the point of attachment of tether 352 to elongate body 340. The weakening will permit the tether to separate from elongate body 340 when a predetermined amount of force is placed on tether 352 after girdle 320 has been placed around the chordae tendinae.

Delivery catheter 310 may be introduced into the left ventricle as described above for system 100. Delivery catheter 310 is advanced to a position to place the distal end adjacent to the chordae tendinae. Secondary catheter 330 is advanced to exit delivery catheter 310. As secondary catheter 330 is advanced, the secondary catheter begins to curve around the chordae tendinae. Continued advancement of secondary catheter 330 completes a loop around the chordae tendinae. Hook 348 may extend out of secondary catheter 330 during deployment. In this embodiment, hook 348 may engage secondary catheter 330 with the completion of the loop therein. Secondary catheter 330 is then retracted to expose girdle 320. As the secondary catheter is retracted, hook 348 engages tether 352. The practitioner then pulls tether 352 in a proximal direction to draw hook 346 into engagement with hook 348, thus forming girdle 320. Once hook 346 is engaged with hook 348, the practitioner exerts a predetermined amount of force on tether 352 to separate the sacrificial joint. Other techniques using deflectable tip catheters or endoscopic manipulation may be used to wrap elongate body 340 around the chordae tendinae and to engage hooks 346 and 348 to form girdle 320. Once in place, girdle 320 draws the chordae tendinae closer together to form a bundle to effectively achieve chordal shortening. This shortening of the chordae tendinae resolves or reduces valve leaflet prolapse.

The advancement of delivery catheter 310 and secondary catheter 330 to and around the chordae tendinae may be monitored by methods known in the art such as fluoroscopy and ultrasonography. In one embodiment, delivery catheter 310 and secondary catheter 330 include radiopaque markers to improve fluoroscopic visualization of the components. Girdle 320 may also include radiopaque markers or the like to improve fluoroscopic visualization.

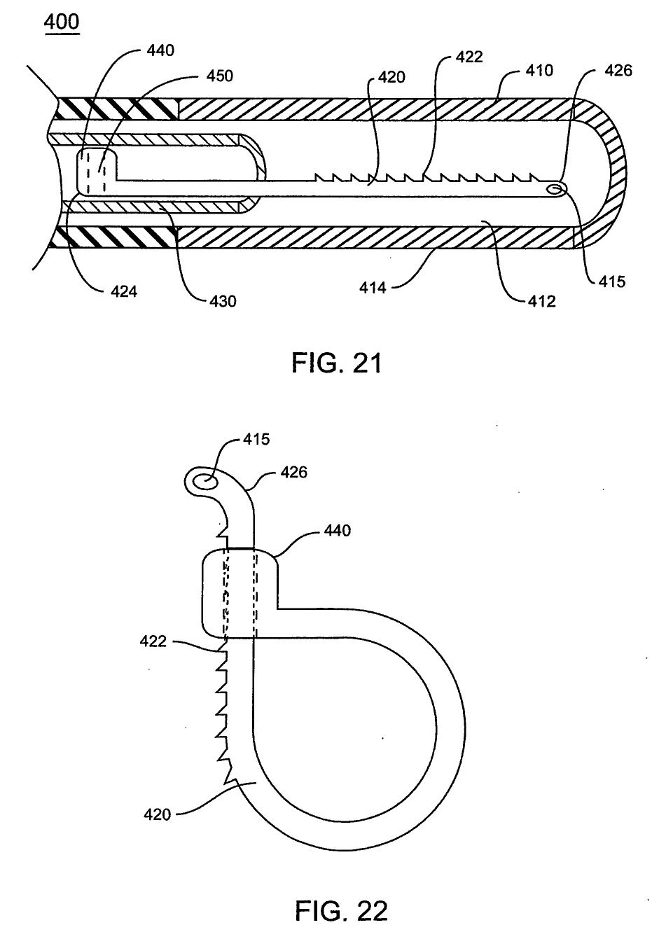

FIGS. 21 and 22 illustrate another embodiment of heart valve repair system 400 made in accordance with the present invention. Heart valve repair system 400 comprises delivery catheter 410, girdle 420 and holding tube 430. Delivery catheter 410 includes lumen 412 and distal end 414. Holding tube 430 is disposed within lumen 412 of delivery catheter 410. Girdle 420 includes a ratchet-type locking mechanism comprising lock portion 440 and at least one tooth 422, or a series of teeth 422. Lock portion 440 is located at proximal end 424 of girdle 420. Lock portion 440 includes lumen 450 for receiving distal end 426 of girdle 420. Teeth 422 are located adjacent distal end 426 of girdle 420. Girdle 420 may also include eyelet 415. Eyelet 415 may comprise an attachment for securing an actuation device (not shown). Girdle 420 may be formed from material similar to those discussed above for girdle 120.

Teeth 422 may comprise a shape-memory material and may be heat set or otherwise shaped into protrusions from the elongate body of girdle 420. As distal end 426 is drawn through lumen 450 of lock portion 440, teeth 422 are deflected in order to fit through the lumen 450. Once proximal to the lock portion 440 and no longer constrained by the lumen 450, at least one of the teeth resumes its preset shape. In an alternative embodiment (not shown), teeth 422 may comprise one indentation or a series of indentations in the body of girdle 420, and lock portion 440 may comprise a mating tang within lumen 450 for engagement with any of the indentations. Teeth 422 and lock portion 440 retaining girdle 420 around the chordae tendinae by preventing girdle 420 from passing back through lock portion 440.

Delivery catheter 410 may be introduced into the left ventricle in a manner as those described above for systems 100 or 300. Delivery catheter 410 may include a deflectable tip, as is known in the art, for positioning and wrapping girdle 420 around the chordae tendinae, and for causing engagement of the locking mechanism. In another embodiment, girdle 420 returns to a pre-curved shape when deployed, inserting distal tip 426 through lock portion 440. An actuating device (not shown) may then engage eyelet 415 and draw tip 426 through lumen 450 to engage the locking mechanism and tightening girdle 420 around the chordae tendinae.

In place, girdle 420 draws the chordae tendinae closer together to form a bundle to effectively achieve chordal shortening. This shortening of the chordae tendinae resolves or reduces valve leaflet prolapse.

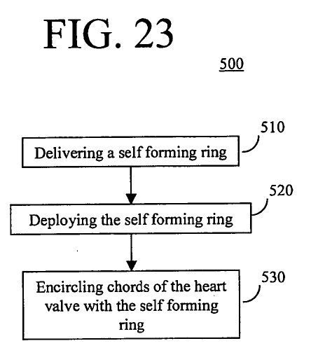

FIG. 23 shows a flow chart for a method 500 of using a heart valve repair system. Method 500 begins by delivering a girdle proximate the chordae tendinae of the heart valve to be repaired (Block 510). The girdle may be delivered by a delivery catheter as is well known in the art. In one embodiment, the elongate delivery element includes a catheter with a lumen and a push rod positioned within the lumen of the catheter. The girdle is held in a deformed linear delivery configuration within the catheter. Once properly positioned, the girdle is released from the catheter (Block 520). The girdle may be extended by pushing the girdle from the catheter using the pushrod. In another embodiment, the catheter forms a retractable sleeve and the push rod acts as a holding device to hold the girdle in a desired position adjacent the chordae tendinae. Then, once positioned properly, the catheter is retracted from the girdle allowing the girdle to be deployed.

During deployment, the girdle encircles the chordae tendinae of the heart valve by transitioning from the linear delivery configuration to the annular treatment configuration. Once fully deployed the chordae are completely encircled (Block 530) whereupon, the girdle forms a bundle of the chordae tendinae to achieve chordal shortening as described above.

It is important to note that FIGS. 1-23 illustrate specific applications and embodiments of the present invention, and are not intended to limit the scope of the present disclosure or claims to that which is presented therein. For example, the heart valve repair system of the present invention can be used for other heart valves in addition to the mitral valve. Different arterial and venous approaches to the valve can also be used. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A girdle for surrounding a plurality of chordae tendinae comprising:
a filamentous body comprising a shape memory material to allow a transition between a linear delivery configuration and an annular treatment configuration.

2. The girdle of claim 1 wherein the shape memory material is a material chosen from a group consisting of: a nitinol alloy, a stainless steel, a cobalt-based alloy, an MP35N® alloy, an Elgiloy® alloy, an engineering plastic, an amide, a polyimide, a polyolefin, a polyester, a urethane, a thermoplastic, a thermoset plastic, and a blend, a laminate and a copolymer of the above materials.

3. The girdle of claim 1 wherein the annular treatment configuration of the girdle has a shape selected from a group consisting of: a ring, a hollow conical frustum, a hollow cylinder, a hollow hourglass, an open coil, a closed coil, and a combination of the above shapes.

4. A system for treating a heart valve comprising:
an elongate delivery catheter having a lumen; and
a girdle having an annular treatment configuration sized and shaped to surround a plurality of chordae tendinae of the heart valve, the girdle having a linear delivery configuration sized and shaped to be releaseably disposed within the lumen of the delivery catheter.

5. The system of claim 4 further comprising a push rod slidably disposed within the lumen of the delivery catheter and being capable of pushing the girdle out of the delivery catheter.

6. The system of claim 5 wherein the push rod includes a flexible distal portion.

7. The system of claim 4 wherein the girdle has a shape memory of the annular treatment configuration to which the girdle tends to reform after a having been deformed to the linear delivery configuration.

8. The system of claim 4 wherein the girdle comprises;
an elongate body having first and second ends; and
a locking mechanism for locking the girdle in the annular treatment configuration.

9. The system of claim 8 wherein the locking mechanism comprises:
a first hook disposed adjacent the first end; and
a second hook disposed adjacent the second end and adapted for engagement with the first hook.

10. The system of claim 8 further comprising:
an elongate tether releasably attached to the girdle.

11. The system of claim 8 wherein the elongate body comprises an elastic material.

12. The system of claim 8 wherein the locking mechanism comprises:
a lock portion disposed at the first end, the lock portion having a lumen for receiving the second end; and
at least one tooth disposed adjacent the second end and adapted for engagement with the lock portion.

13. A method for treating a heart valve, the method comprising:
delivering a girdle in a lumen of a catheter adjacent the heart valve;
releasing the girdle; and
encircling a plurality of chordae tendinae of the heart valve with the girdle.

14. The method of claim 13 wherein delivering the girdle comprises positioning the catheter proximate a plurality of chordae tendinae of the heart valve.

15. The method of claim 13 wherein delivering the girdle in a lumen of a catheter comprises inserting the catheter percutaneously.

16. The method of claim 13 wherein the catheter is inserted percutaneously and advanced transluminally to a left ventricle through an aortic valve.
